(19) United States
(12) Patent Application Publication
(10) Pub. No.: US 2024/0382343 A1
(43) Pub. Date: Nov. 21, 2024

(54) DEVICE FOR DIAGNOSING OCULAR SURFACE DISEASES AND KIT FOR THE ADMINISTRATION OF TOPICAL OCULAR THERAPIES BY MEANS OF AEROSOL

(71) Applicants: Salvatore CRUGLIANO, Legnano (MI) (IT); Antonio DI ZAZZO, Anzio (RM) (IT); Carlo FRONTINI, Castellanza (VA) (IT); Emillio COMPAGNONE, Wasquehal (FR); Emiliano SCHENA, Roma (RM) (IT); Antonella GRASSO, Ariano Irpino (AV) (IT); Paolo DI ZAZZO, Pozzilli (IS) (IT)

(72) Inventors: Salvatore CRUGLIANO, Legnano (MI) (IT); Antonio DI ZAZZO, Anzio (RM) (IT)

(73) Assignees: Salvatore CRUGLIANO, Legnano (MI) (IT); Antonio DI ZAZZO, Anzio (RM) (IT); Carlo FRONTINI, Castellanza (VA) (IT); Emilio COMPAGNONE, Wasquehal (FR); Emiliano SCHENA, Roma (RM) (IT); Antonella GRASSO, Ariano Irpino (AV) (IT); Paolo DI ZAZZO, Pozzilli (IS) (IT)

(21) Appl. No.: 18/691,976

(22) PCT Filed: Sep. 5, 2022
(86) PCT No.: PCT/IB2022/058336

§ 371 Date: Mar. 14, 2024

(30) Foreign Application Priority Data
Sep. 16, 2021 (IT) 102021000023840

(51) Int. Cl. A61F 9/00; A61M 11/02

(52) U.S. Cl. CPC A61F 9/0008; A61M 11/02; A61M 2205/3306; A61M 2205/35; A61M 2205/36; A61M 2205/502; A61M 2209/06; A61M 2210/0612; A61M 2240/00

(57) ABSTRACT

A device for diagnosing ocular surface diseases is provided which includes a diagnostic terminal provided with eyepiece cups and sensors capable of detecting information from the ocular surface, connected to a processing base capable of processing data and sending it to a server. The invention uses a kit for the administration of therapies which includes a therapeutic terminal provided with eyepiece cups capable of delivering medicines in the form of an aerosol and a control base provided with aerosol, digital screen and Internet connection.

### FIELD OF THE INVENTION

The present invention relates to the definition of the pathological picture and of the treatment of patients suffering from ocular pathologies by proposing an innovative diagnostic tool together with a means for administering the appropriate therapies based on aerosols.

### PRIOR ART

Dry eye disease is a chronic disorder of the ocular surface of multifactorial origin, which severely limits the patients quality of life. Today it represents one of the most frequent eye diseases in the Western world. The clinical picture manifests itself with ocular discomfort in the initial stages and can extend to corneal damage with risk of ulceration and perforation, and to irreversible scarring of the ocular surface. Dry Eye Disease (DED) is more frequent with advancing age and is a consequence of systemic diseases such as rheumatic, degenerative and hormonal diseases. Such condition can also be caused by chronic local and systemic therapies, while exposure to environmental factors, such as wind, air conditioning, excessive use of contact lenses and video screens are factors that aggravate or trigger the disease.

Currently, dry eye is defined as a “multifactorial disease of the ocular surface characterized by a loss of homeostasis of the tear film, and accompanied by ocular symptoms, in which tear film instability and hyperosmolarity, ocular surface inflammation and damage, and neurosensory abnormalities play etiological roles” (Dews II, 2017).

Therefore it is considered a real disease that affects the complex system of the ocular surface, which is based on the interaction of different tissues such as the lacrimal glands, the eyelids, the cornea and the conjunctiva, but above all the tear film, the local immune system and the neuroendocrine one localized in the mucous membranes. The fundamental feature that gives rise to dry eye is a change in the quantity and composition of the tears, which become denser (lacrimal hyperosmolarity) due to a reduced production of the liquid component by the main lacrimal gland or due to excessive evaporation. The increase in osmolarity in turn causes damage to the epithelial cells of the conjunctiva and the cornea, as well as the goblet cells that produce the normal mucous component of tears, and induces an inflammatory reaction of the entire ocular surface. These alterations trigger a vicious circle that aggravates the dryness situation and causes the process to become chronic.

To date, the diagnosis of dry eye is an “unmet need” of ophthalmology, as all the tests used for the diagnosis are surrogates. The clinical suspicion is confirmed by the evaluation of the ocular surface with the slit lamp and, more particularly, by the finding of a reduced thickness of the lacrimal menisci, that is the layer of tears present between the eyelid rim and the surface of the eyeball. The diagnosis is then clarified by some tests: the evaluation of the break up time (BUT) of the tear film, that is the period of time in which dry areas are formed on the surface of the cornea between one blinking and another; the Schirmer test, which evaluates the length of the soaked portion of a strip of paper inserted in the conjunctival fornix, between the lower eyelid and the eye, over a certain period of time, generally 5 minutes; the tear osmolarity test, which allows a quantitative assessment of the degree of alteration of the tears; and the evaluation of the conditions of the epithelial surface with the help of particular colors (fluorescein, lissamine green, rose bengal).

The object of the present patent is to propose a minimally invasive, rapid, innovative but above all all-encompassing diagnostic method for dry eye disease, based on a device capable of detecting, by means of sensors, the humidity and temperature of the ocular surface, of connecting to a remote device for data processing and possibly of administering an appropriate therapy according to the pathological picture detected. The device which is the subject of the present industrial patent application is therefore a complete system for dry eye diagnosis, prognosis, treatment and follow-up, which can also be used in the diagnosis and treatment of other (infectious and/or inflammatory) diseases of the ocular surface and in the detection of imported biometric data such as glucose concentration. In fact, such device allows not only the qualitative diagnosis of the type of dry eye but also the quantitative one with a single examination, combining diagnostic, prognostic and staging capabilities. As explained below, the two elements considered, namely the diagnostic and the therapeutic, can be used jointly in a single system or structured as two separate entities.

### Description of the Invention

According to the present invention, a device for diagnosing ocular surface diseases and a kit for administering the relative therapies by means of aerosols, capable of effectively solving the above problems, are provided. Specifically, the diagnostic device comprises at least an ergonomic terminal with eyepiece cups connected to a data processing base, and can be used both in the diagnosis and in the classification of dry eye syndrome, and in the evaluation of therapeutic progress. The ergonomic terminal with diagnostic functions is designed to be placed in contact with the patient's face and can be roughly shaped like a helmet, a special eyewear with rear support or a gun held in the operator's hand by means of a handle, having in all cases disposable eyepiece cups or made of sterilizable material which have the purpose of bringing the sensors in the vicinity of the patient's eyeball in a closed and controlled environment.

The eyepiece cups can be of the conventional type or modified for the collection of tear discharge when the humidity reaches 100%, moreover, they can have an absorbent component in contact with the patient's eyelid skin covered with a waterproof layer, to eliminate interference due to sweating. They can be fixed or removable, so that the most suitable sensor is associated with a specific cup.

The sensors placed in the eyepieces of the ergonomic diagnostic terminals can be hygrometer and thermometer, often integrated in a single sensor, suitable to measure relative humidity and temperature, relative humidity (RH) in a given environment is defined as ρω/ρs×100%, where ρω is the water vapor density and ρs is the water vapor density at saturation. Using this sensor, it is possible to collect elements useful for diagnosis and define the difference between those affected by the disease and the normal population.

Other sensors to be applied to the eyepiece cups for the detection of data from the surface of the eye can be: sensors used for the evaluation of the osmolarity/osmolality of the tear discharge; and sensors for contact tear production, sensitive to the progressive imbibition of a support. To all these elements stimuli such as hot, cold or room temperature air, induced change in the humidity rate inside the cup and/or a light stimulus to be stared at can be associated.

It is also possible to insert a micro-camera in the eyepiece cups to evaluate the frequency and dynamics of blinking during the execution of the tests in basal conditions or under stimulus. Furthermore, the system can be implemented with the collection of tear discharge and accurate analysis thereof by means of spectrometry, spectrophotometry or rapid evaluation by colorimetric strips (such as detection of leukocytes, nitrites, PH, glucose concentration, etc.). A further data of diagnostic interest could be that relating to the perception of pain/discomfort felt by the patient both in basal condition and under stimulus. To detect this data and transform it into a digital signal useful for subsequent processing, it could be advantageous to position a pressure sensor at a button mounted on an ergonomic support, this being connected by means of a cable or other wireless system to the processing base or mounted on the processing base itself, through which the patient, when questioned and/or when subjected to a stimulus, expresses his/her assessment of pain/discomfort perceived by pressing the button with higher/lower intensity.

All the data collected by the ergonomic diagnostic terminals, and possibly by the aforementioned optional button for pain assessment, are sent to a data processing base, connected in wired or wireless mode, which stores the data (at any time usable by the operator) and processes the information by applying Artificial Intelligence algorithms such as, by way of non-limiting example, computational statistics, pattern recognition, adaptive filtering, image processing etc., with the aim of providing useful tools to the doctor for a correct diagnosis and treatment of detected pathologies. Such processing base is in turn connected to an external server to which it transmits aggregate data for any further subsequent processing. In a variant, the processing base can be replaced by non-dedicated mobile devices (smartphones, tablets, laptops) or fixed devices (PCs), connected in wired or wireless mode to the diagnostic terminal, suitably provided with hardware capable of supporting adequate dedicated software.

In addition to the diagnostic device, the present invention provides a kit capable of administering any topical ocular therapy normally instillable through eye drops, with the advantage, compared to the latter, of overcoming the missed administration due to blinking, of being able to vary at will the concentrations of the drugs used and of being particularly suitable for uncooperative patients, such as infants or people with disabilities. Such kit comprises a therapeutic terminal implemented by means of a suitably modified aerosol mask, connected to a control base provided with an aerosol device, and a heat source can be associated with it, such as a thermoregulated resistance or an ultrasound emitter, both useful in dry eye syndrome therapy.

Furthermore, the kit can comprise an internal ionizer with the purpose of activating the active ingredients, maximizing the effect thereof and/or facilitating the achievement of the site of action at the level of the entire visual apparatus, allowing to store the drugs in a non-inactive form until the moment of administration, this guarantees the possibility of storing the medicines for a longer time. The above is also valid in the case in which nanoparticles are administered for therapeutic and/or diagnostic purposes.

### DESCRIPTION OF THE FIGURES

The foregoing advantages, as well as other advantages and features of the present invention, will be illustrated with reference to the accompanying drawings, which are to be considered purely illustrative and not limiting or binding to the effects of the present patent application, in which

FIG. 1 shows a side and rear view of a possible embodiment of a single-sensor “gun” diagnostic terminal 100, where the eyepiece cup 10, the sensor 20 placed inside it, a processing board 30 provided with a processor 31, memory 34 and wireless connection device 32, with the optional connection cable 33, a digital screen 40 with buttons 41 and a rechargeable power supply battery 50 arranged in the handle are visible;

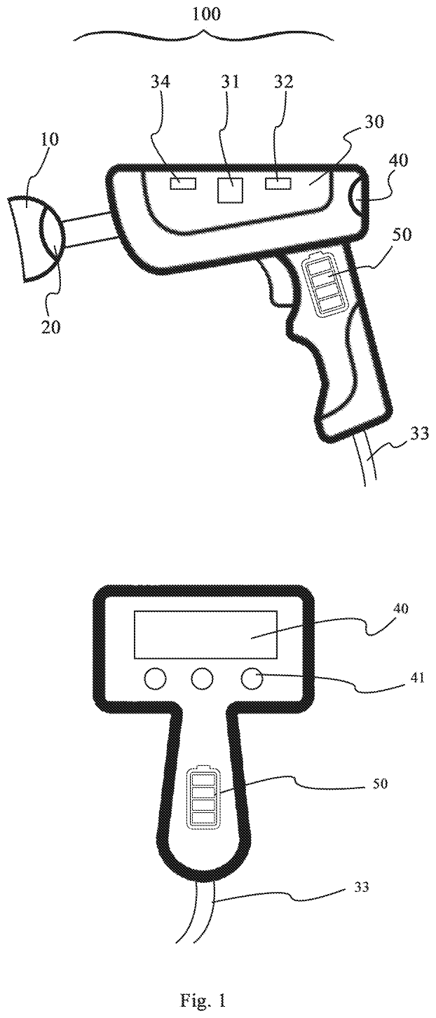

FIG. 2 shows a possible embodiment of the processing base 200 where a digital screen 80, menu buttons 90, an optional printer 70, a power supply cable 95, a connection cable 33 which connects to the diagnostic terminal 100, are visible, said base 200 being managed by a processing board 60 having a processor 61, a memory 62 and a remote connection device 63 capable of connecting to the Internet and/or to any remote diagnostic and/or therapeutic terminals;

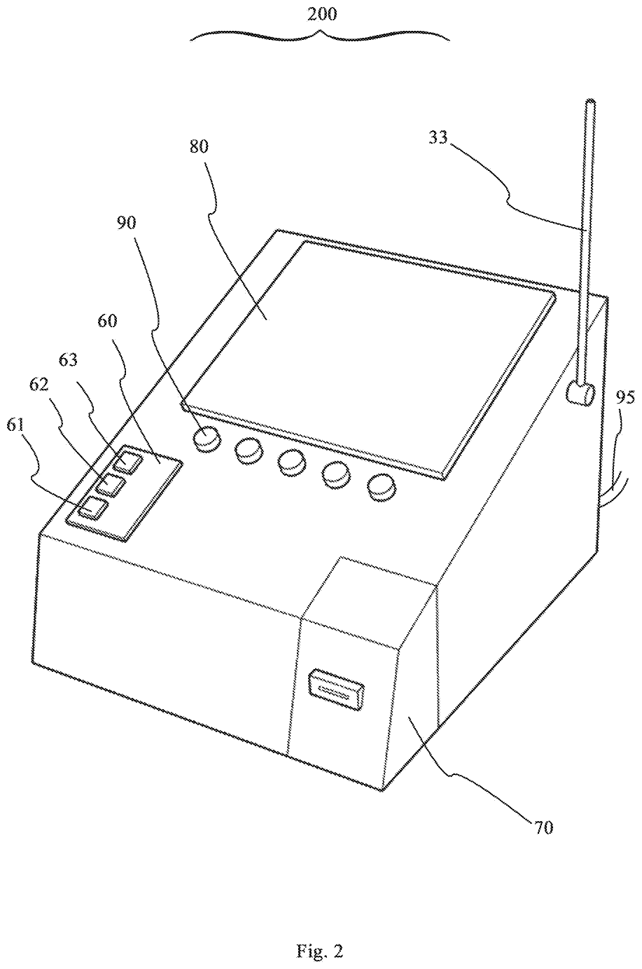

FIG. 3 shows a possible embodiment of the control base 300 for administering the therapy, provided with a computer 330, a display 310, buttons 320, an aerosol device 350 and connected to a therapeutic terminal 400 with an eyepiece dispensing cup 410 and a thermoregulated resistance or other system for varying the temperature of the aerosol 420 in a controlled manner, an ionization system (ionizer) 430;

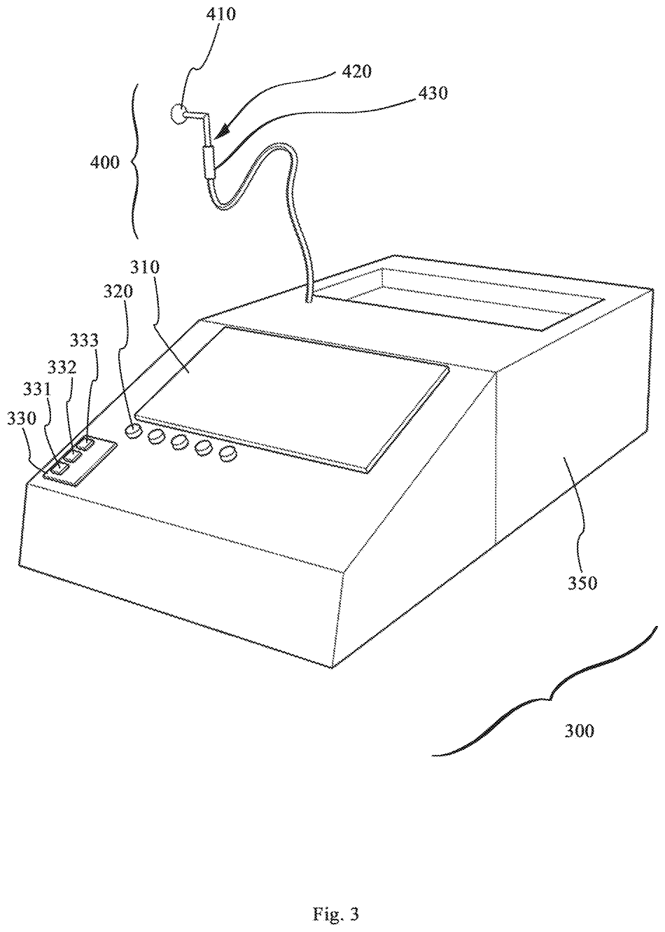

FIG. 4 illustrates a possible operating diagram of the device as a whole, where a diagnostic terminal 100 sends the information detected by the ocular surface to a processing base 200 which processes it and sends it to a remote server 500, from which it is downloaded on a control base 300 which is capable of delivering therapy through a therapeutic terminal 400.

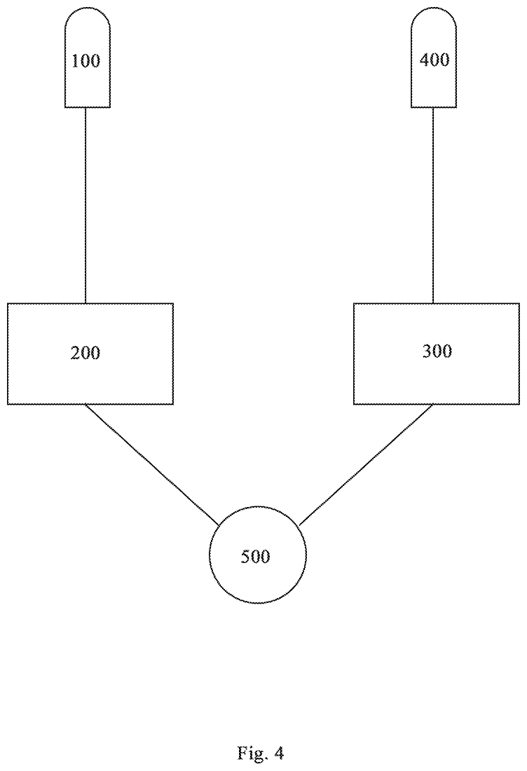

### DETAILED DESCRIPTION OF THE INVENTION

It will be immediately apparent that innumerable variations and modifications (for example relating to shape, dimensions, arrangements and parts with equivalent functionality) may be made to what has been described without departing from the scope of the invention as appears in the appended claims.

The present invention will now be illustrated, purely by way of non-limiting or binding example, as a diagnostic terminal 100 connected to a processing base 200 by means of a wireless connection, the sensor 20 being installed in the removable eyepiece cup 10 positioned on said terminal 100, a hygrometer/thermometer capable of detecting the relative humidity generated by tear evaporation from the ocular surface. The data detected by the sensor are shown in real time on the digital screen 40 present on the diagnostic terminal 100 for the convenience of the operator, then they are transmitted, by wireless connection, to the processing base 200 which stores and processes them for subsequent integration with previous data, both from the same measurement and from previous measurements, through the application of artificial intelligence algorithms. Said processing base 200 being also able to print them by means of a suitable optional printer 70. The digital data thus integrated and processed are useful for the diagnosis and possible processing of a therapy and are sent to a remote server 500 through an Internet connection, on said server 500 they are also integrated by means of artificial intelligence with a database consisting of data collected from similar devices in use in other locations and are unloaded onto a control base 300 for administering the therapy. Said base 300 displays the prescribed therapy by means of a screen 310 and then delivers the medicine, by means of an integrated aerosol 350, through the therapeutic terminal 400 provided with a suitable eyepiece cup 410, a thermoregulated resistance 420 or other system for varying the temperature of the aerosol, such as an ultrasound emitter, and with an ionization system of the active ingredient, nanoparticle and/or therapeutic substance to be delivered.

Alternatively, the therapeutic data obtained from the processing base 200 can be directly sent to the control base 300. Said data can be viewed by the operator who has complete access and possibility of modification and/or integration at his/her discretion.

It is clear that modifications, additions or variants may be made to the invention described thus far which are apparent to those skilled in the art, without departing from the scope of protection that is provided by the appended claims.

1. A device for diagnosing ocular surface diseases, comprising:
at least a processing base provided with a screen, push button panel, and an electronic board having a processor, a memory and a remote connection device, said processing base being suitable to receive and process the data coming from a sensor arranged on a diagnostic computer and said processing base being provided with dedicated software capable of applying, to said data, appropriate artificial intelligence algorithms such as, by way of non limiting example, computational statistics, pattern recognition, adaptive filtering, image processing algorithms etc., with the aim of providing tools useful to the physician for a correct diagnosis and treatment of the detected diseases;
at least a diagnostic computer comprising: a removable eyepiece cup made of sterilizable material; at least a sensor suitable to detect data from the ocular surface; an electronic board having a processor, memory and a remote connection device; rechargeable battery for power supply, and a set of buttons; said diagnostic computer potentially being made with a single sensor and a single eyepiece cup in the form of a “gun” with handle, or with dual sensor having two distinct eyepiece cups (mounted on a helmet for the head of the patient, or with dual sensor having two distinct eyepiece cups mounted on a eyepiece with an extensive eye support for the nape.

2. The device, according to the claim 1, wherein the remote connection device present on the processing base sends the results of the processing carried out on the data obtained from the diagnostic computer to a remote server, said data being processable subsequently, i.e. shared, or downloaded from said control base, suitably identified by means of a unique alphanumeric code, for the subsequent administration of the therapy.

3. The device according to claim 1, wherein the processing base consists of a non dedicated external mobile or fixed device, such as a fixed or portable computer, a mobile phone, a tablet or other remote device, suitably provided with hardware capable of supporting a dedicated software and connected to the diagnostic computer in wired or wireless mode.

4. The device according to claim 1, wherein the diagnostic computer is provided with a digital screen (40) for the immediate display of the datum detected by the sensor, in order to allow the operator a better understanding of the diagnostic datum detected.

5. The device according to claim 1, wherein said digital screen present on the processing base allows the operator to continuously display the diagnostic data, said continuous display of data, also being allowed on the other screen on the diagnostic computer.

6. The device, according to claim 1, wherein said eyepiece cup is suitably modified for the collection of the tear discharge when the relative humidity detected by the sensor reaches 100%; said tear discharge potentially being subsequently examined by means of a spectrometer or spectrophotometer preferably installed on said processing base and the data detected by the processing being integrable in the overall processing of the diagnostic data by means of said artificial intelligence algorithms.

7. The device, according to claim 1, wherein the sensor positioned inside the eyepiece cup on the diagnostic computer is a sensor used to evaluate the osmolarity/osmolality of the tear discharge.

8. The device, according to claim 1, wherein the sensor positioned inside the eyepiece cup on the diagnostic computer is a hygrometer/thermometer sensor suitable to detect relative humidity and temperature in the environment created around the surface of the eye by the eyepiece cup.

9. The, according to claim 1, wherein the processing base is connected to a button provided with a pressure sensor, suitable to detect the pain/discomfort felt by the patient in basal condition and/or under stimulus; the data detected by said button being sent to the processing board positioned on said base for subsequent integration and processing; said button being mounted directly on the processing base or on a handle or other ergonomic support to be handed to the patient and suitably connected to the processing base.

10. The device, according to claim 1, wherein the processing base is provided with a printer suitable to print medical receipts containing the detected data, the assigned therapies and/or other information useful for diagnosis and therapy according to the judgment of the operator.

11. The device diseases, according to claim 1, wherein the eyepiece cup mounted on the diagnostic terminal is provided with a micro camera, positioned next to the sensor or in another position inside said eyepiece cup, suitable to evaluate the frequency and dynamics of the blinking and closing of the eyelid when running the tests in basal conditions and under stimulus; the video of said micro camera being shown on the digital screen present on said processing base.

12. The device, according to claim 1, wherein the eyepiece cup mounted on the diagnostic computer is provided with a device, positioned next to the sensor or in another position inside said eyepiece cup. suitable to dispense air blows at various temperatures, suitable to run diagnostic tests on the ocular surface.

13. The device, according to claim 1, wherein the eyepiece cup mounted on the diagnostic computer is provided with a device, positioned next to the sensor, suitable to emit a luminous stimulus at various intensities. suitable to run diagnostic tests on the ocular surface.

14. The device diseases, according to claim 1, wherein the eyepiece cup mounted on the diagnostic computer is provided with a device, positioned next to the sensor, suitable to induce a variation in the humidity level of the environment inside said eyepiece cup, with the purpose of running diagnostic tests on the ocular surface.

15. The device, according to claim 1, wherein said diagnostic computer and said processing base are connected by a cable suitable to transmit data and/or recharge the battery positioned on the handle of said computer.

16. A kit for the administration of topical ocular therapies by means of aerosol, comprising:
a control base provided with a screen, push button panel, aerosol device and electronic board having a processor, memory and remote connection device, said control base being suitable to administer any topical therapy avoiding non administration due to blinking, being suitable to vary the concentrations of the drugs used at will and being particularly suitable for non collaborative patients, such as new borns or persons with disabilities;
a therapeutic computer suitable to deliver the prescribed medicinal product as therapy following the diagnosis carried out with the device of any one of the preceding claims or by direct entry of therapeutic data by the operator in the control base, connected to said control base and comprising an eyepiece cup to be brought into contact with the face of the patient and a thermoregulated resistor or other aerosol thermoregulation system.

17. The kit for the administration of topical ocular therapies by means of aerosol, according to claim 16, wherein the therapeutic computer connected to the control base is provided with a system for ionizing the drug, nanoparticle and/or therapeutic substance to be dispensed, in order to activate the active ingredients, maximize the effect thereof and/or facilitate the reaching of the site of action at the level of the whole visual apparatus, allowing to store the drugs in a non active form until the moment of administration.

18. A device for diagnosing ocular surface diseases and kit for the administration of topical ocular therapies by means of aerosol according to claim 1, wherein the processing bases and the control bases are integrated in a single diagnostic/therapeutic device or they can be distinct and separate but capable of being connected through a wireless connection for the direct exchange of diagnostic data, treatments and therapies useful to both the patient and the operator.
